(19) United States
(12) Patent Application Publication
(10) Pub. No.: US 2015/0088083 A1
(43) Pub. Date: Mar. 26, 2015

(54) Hand Pan TM

(71) Applicant: Timothy Graham Allen, Piedmont, SC (US)

(72) Inventor: Timothy Graham Allen, Piedmont, SC (US)

(21) Appl. No.: 13/947,494

(22) Filed: Sep. 22, 2013

(51) Int. Cl. A61M 3/02; A61B 19/00

(52) U.S. Cl. CPC A61M 3/0287; A61B 19/38; A61B 19/42
USPC 604/356

(57) ABSTRACT

The Hand Pan™ is an improvement on existing plastic basins used for medical purposes. It is specifically designed and shaped to neatly accommodate the irrigating of hand wounds (traumatic and surgical) in the operating room and emergency room settings. Nothing else currently exists like it.

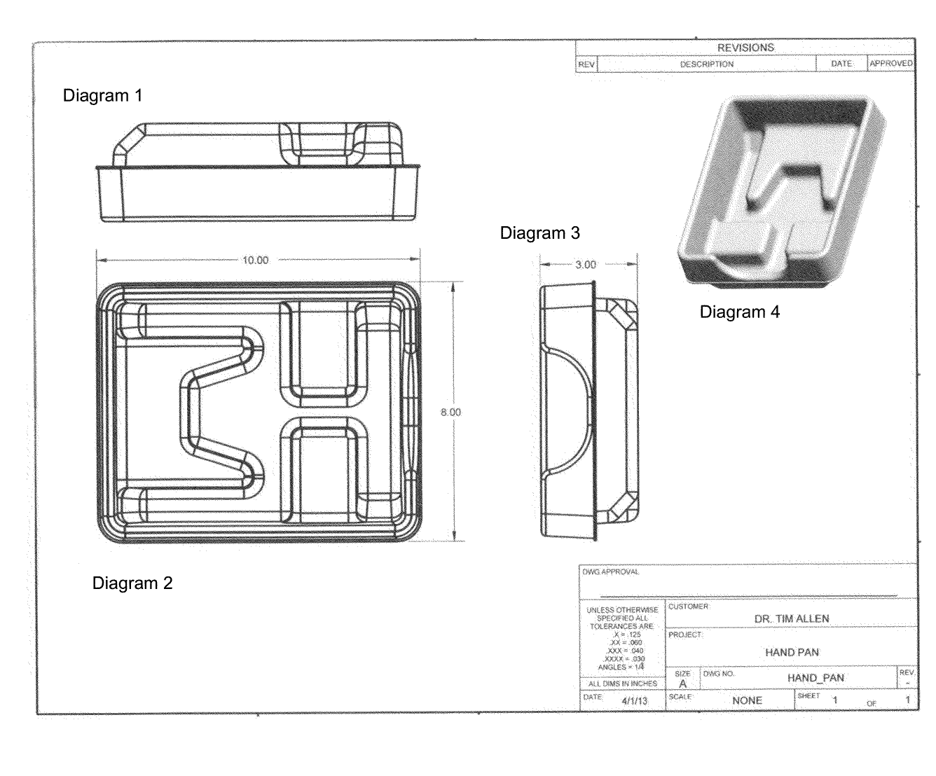

The Hand Pan™ is a unique medical product that falls into the category of irrigation basin. It is specifically designed to accommodate the human hand and wrist so that a traumatic or surgical wound, can be safely and neatly irrigated to cleanse it of blood, tissue, soilage, infection, and/or other contamination or debris. Because it is designed to hold the hand and wrist safely and securely during the procedure, it protects the sterile field during irrigation and prevents spillage of irrigation fluid onto the surrounding area as well as the health care provider, thereby helping to prevent exposure of the worker to biologic contamination. There is nothing else that I am aware of on the market available to adequately accomplish this task.

The Hand Pan™ is made by standard suction plastic molding techniques, and thus the manner of manufacturing the product is not new and is well known. The justification for patent comes from the design of the product and the specific use for which it is intended (as described above). Nothing else currently exists for this usage or with this type of design.

### DRAWINGS DESCRIPTION PAGE

Drawing 1: Side view of invention.

Drawing 2: Top down view of invention.

Drawing 3: End view of invention.

Drawing 4: 3-dimensional rendering of invention.

1. To my knowledge there is no other device or product in existence which is designed and manufactured to accomplish the purpose and use of the Hand Pan™ as outlined in the preceding Specification. I am the sole designer and inventor of the Hand Pan™ as described and depicted in this application.
